(19) United States
(12) Patent Application Publication
(10) Pub. No.: US 2023/0397799 A1
(43) Pub. Date: Dec. 14, 2023

(54) Leakage Test Connector

(71) Applicant: Advanced Sterilization Products, Inc., Irvine, CA (US)

(72) Inventor: Nick N. Nguyen, Irvine, CA (US)

(73) Assignee: Advanced Sterilization Products, Inc., Irvine, CA (US)

(21) Appl. No.: 18/036,225

(22) PCT Filed: Nov. 9, 2021
(86) PCT No.: PCT/IB2021/060367

§ 371 Date: May 10, 2023

Related U.S. Application Data
(60) Provisional application No. 63/112,075, filed on Nov. 10, 2020.

(51) Int. Cl. A61B 1/00

(52) U.S. Cl. CPC A61B 1/00128; A61B 1/00119; A61B 1/00057

(57) ABSTRACT

A leakage test connector may comprise a male connector having an outer circumferential surface, a female socket connector that mates to the male connector, and an O-ring mounted about an inner circumferential surface of the fluid passage of the female socket connector. The male connector lacks an O-ring on the outer circumferential surface in an unmated state with the female socket connector. The male connector may also comprise a valve actuator pin, while the female socket connector comprises a pin that depresses the valve actuator pin. The valve actuator pin includes a top surface that is disposed flush with a tip of the male connector to minimize the likelihood of contamination on the male leakage connector.

### FIELD

The subject matter disclosed herein relates to a leakage test connector and, more specifically, a leakage test connector comprising a male connector assembly and female connector assembly, used for testing leakage in endoscopes.

### BACKGROUND

Endoscopes are reusable medical devices generally used in minimally invasive procedures. Endoscopes are flexible instruments, which are costly, complex and fragile instruments. After use, the endoscope can be reprocessed, i.e., decontaminated, to avoid causing infection or illness in a subject.

Typically, endoscope reprocessing is performed by a disinfection procedure that includes at least the following steps: removing foreign material from the endoscope, cleaning the endoscope, and disinfecting the endoscope by, among other things, submerging it in a disinfectant capable of substantially killing microorganisms thereon, e.g., infection causing bacteria. Endoscope reprocessing may be conducted by a healthcare worker or with the assistance of machinery, such as an Endoscope Cleaner and Reprocessor (ECR), e.g., the EVOTECH® Cleaner and Reprocessor manufactured by Advanced Sterilization Products, Inc. of Irvine, California.

Endoscopes are often leak tested prior to reprocessing to ensure that cleaning and disinfecting liquids do no penetrate into areas of endoscopes that can damage the endoscopes. Such leak tests may assist in: 1) confirming the presence and proper body connection to an endoscope; 2) assessing whether the endoscope body has a leak rate beyond the ability of the system to maintain sufficient body pressure in assuring subsequent processing can be performed without moisture ingress; 3) checking the endoscope body pressure and its structural integrity; and 4) stabilizing the endoscope scope body pressure during the entire cycle. The EVOTECH® Cleaner and Reprocessor, for example, includes a leakage test subsystem comprising a leakage connector extending into its endoscope basin.

If any leaks are found, the leakage test subsystem will give an alarm and the cycle will stop. If no leaks are found, the endoscope body is pressurized and once the pressure is stabilized, a vent valve is opened to decay the pressure. When the pressure in the endoscope is increased and the initial stabilization pressure exceeds a certain pressure, the vent valve is opened to reduce the stabilization pressure. The leakage test subsystem makes sure that this pressure is maintained throughout the entire cycle.

According to ISO 15883-1, leakage test subsystems in automated disinfectors are to include a leakage connector that cannot be connected to the typically male connectors of endoscope channels, e.g., the air and water channels. Thus, leakage connectors often also comprise male connectors.

### SUMMARY OF THE DISCLOSURE

A leakage test connector may comprise a male connector having an outer circumferential surface, a female socket connector that mates to the male connector, and an O-ring mounted about an inner circumferential surface of the fluid passage of the female socket connector. The male connector lacks an O-ring on the outer circumferential surface in an unmated state with the female socket connector. Upon mating the male connector to the female socket connector, the O-ring of the female connector contacts the outer circumferential surface of the male connector. The male connector may also comprise a valve actuator pin, while the female socket connector comprises a pin that depresses the valve actuator pin. The valve actuator pin includes a top surface that is disposed flush with a tip of the male connector.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts a perspective view of a leakage test connector;

FIG. 2 depicts a perspective view of a male leakage test connector of leakage test connector shown in FIG. 1;

FIG. 3 depicts an exploded view of male leakage test connector;

FIG. 4A depicts a plan view of a disposable female socket connector of leakage test connector shown in FIG. 1;

FIG. 4B depicts a cross-sectional view of disposable female socket connector along line A-A of FIG. 4A;

FIG. 5A depicts a partial cross-sectional view of an valve actuator pin of male leakage test connector;

FIG. 5B depicts a top down view of valve actuator pin of male leakage test connector;

FIG. 6 depicts a cross-sectional view of an alternative valve actuator of male leakage test connector;

FIG. 7 depicts an exploded view of disposable female socket connector and hose assembly; and

FIG. 8 depicts leakage test connector fixed to an exterior surface of a basin and connected to an endoscope.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

The subject matter disclosed herein relates to a leakage test connector and, more specifically, a leakage test connector for testing leakage in endoscopes. The leakage test connector includes a male leakage test connector with a valve actuator pin used to actuate an internal valve. A top surface of the valve actuator pin is flush with a tip of male leakage test connector and is designed to move up and down to either close the internal valve or actuate (open) the internal valve, respectively. This actuator pin, being flush with a tip of male leakage test connector, reduces any exposed surfaces and, hence, eliminates or reduces the likelihood of contamination on male leakage connector. More specifically, valve actuator pin will avoid or at least minimize liquid present on male connector, which could otherwise end up inside the internal endoscope body during leakage testing.

In addition, the leakage test connector lacks an O-ring groove or O-ring on its outer circumference; instead, an O-ring is installed in a groove on an inner circumference of an off-the-shelf and disposable female socket connector. In this way, when the O-ring degrades, a new female connector may be obtained with a new O-ring to replace the older female connector with the worn O-ring. In those endoscope processors where an O-ring is disposed on the outside of the male leakage connector, when the O-ring becomes worn, a technician must typically be called to replace the O-ring. This O-ring, along with other O-rings in the endoscope reprocessor, can also be color coded to identify the connection types as illustrated in Table 1 below (which are non-limiting, illustrative examples).

TABLE 1

| Color of O-ring | Tube Color Endoscope Connection Type |
|---|---|
| Yellow | Air |
| Blue | Water |
| White | Auxiliary Water, CO2, Forward Water Jet, |
| | Balloon Fill, Balloon Drain, Lens Rinse, |
| | Suction Valve Housing |
| Green | Auxiliary Water, CO2, Forward Water Jet, |
| | Balloon Fill, Balloon Drain, Lens Rinse |
| Gray | Auxiliary Water, CO2, Forward Water Jet, |
| | Balloon Fill, Balloon Drain, Lens Rinse, |
| | Suction Valve Housing, Elevator |
| Brown | Suction |
| Orange | Biopsy/Instrument Channel/Working Biopsy 1 |
| Clear | Biopsy/Instrument Channel/Working Biopsy |
| | 2, Suction Valve Housing |
| Red | Leak Test |

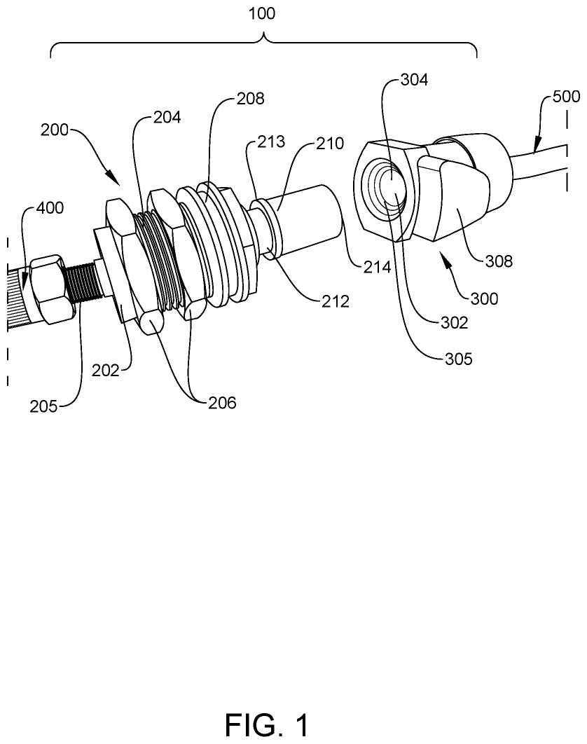

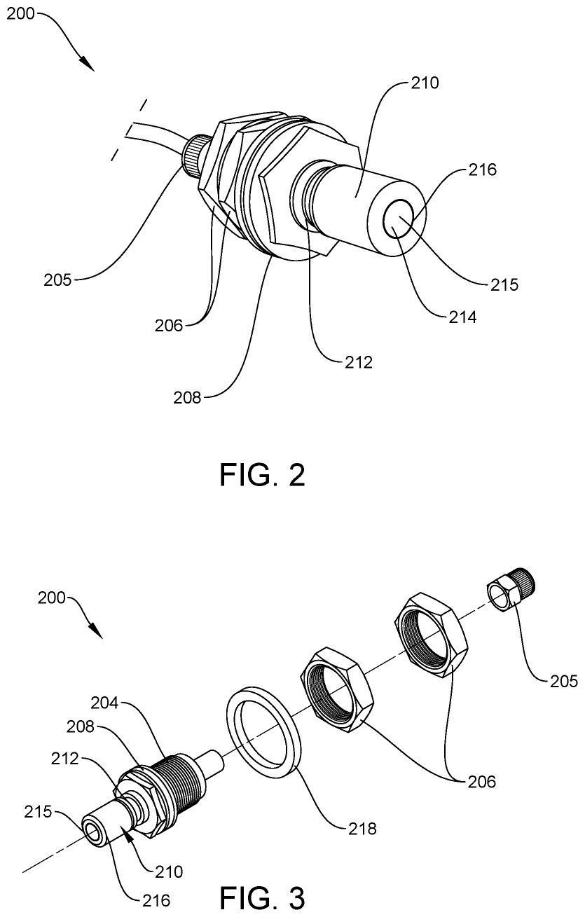

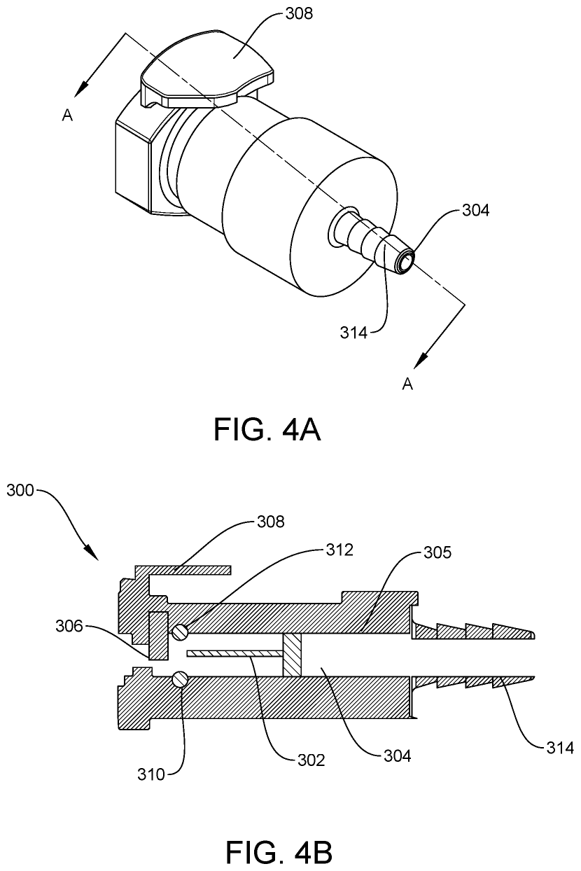

FIG. 1 depicts a perspective view of a leakage test connector 100 comprising a male leakage test connector 200 and a disposable female socket connector 300. FIG. 2 depicts a perspective view of male leakage test connector 200 and FIG. 3 depicts an exploded view of male leakage test connector 200. FIGS. 4A and 4B depict a respective plan view and a cross-sectional view of female socket connector 300.

Referring more specifically to FIGS. 1-4B, leakage test connector 100 comprises male leakage test connector 200 and female socket connector 300. Female socket connector 300 can be a disposable unit, and is structured to mate to male leakage test connector 200 as described further below.

Male leakage test connector 200 comprises a housing 202. Housing 202 may include a threaded portion 204 and a flange 208 to assist in connecting male leakage test connector 200 to a basin of an endoscope reprocessor. At least one securing feature, e.g., nut 206, may be engaged with threaded portion 204 for the same purpose. Nuts 206 may be positioned on an inner surface of the basin and flange 208 may be positioned on an outer, exposed surface of the basin. The flange 208 will prevent housing 202 from completely passing through opening of the basin, in addition to assisting in securing housing 202 to the basin. For example, by fastening or tightening nut(s) 206, male leakage test connector 200, e.g., housing 202, will be secured to the basin with assistance of flange 208 contacting the interior surface of the basin. As shown in FIG. 3, for example, a washer 218 may be used to ensure a watertight seal of housing 202 to the basin.

Male leakage test connector 200 further includes a male connector portion 210 comprising a valve actuator pin 214. A top surface (e.g., tip) 215 of valve actuator pin 214 sits flush to a top surface (e.g., tip) 216 of male connector portion 210 (see, e.g., FIGS. 2, 3 and 5A). As described in more detail below, valve actuator pin 214 is depressed or opened by pin 302 extending from a fluid channel 304 (e.g., hollow portion) of female socket connector 300. For example, upon actuation or depression thereof, valve actuator pin 214 permits testing fluid (e.g., air or liquid) to pass to an endoscope for leakage testing.

As valve actuator pin 214 is flush with top surface 216 of male connector portion 210, it will not be prone to accumulation of water or contamination on a surface thereof. That is, valve actuator pin 214 will not have an exposed surface extending from male connector portion 210 which would otherwise accumulate water or contamination on its surface. This is in contrast to conventional valve actuators that extend beyond a tip of the male connector, allowing accumulation of water or contaminants to sit on an exposed surface of the valve actuator.

In addition, male connector portion 210 is devoid of any O-ring or seal member on its outer surface. Instead, as shown in FIG. 4B, O-ring 310 is provided about an interior circumferential surface 305 of female connector portion 304 of female socket connector 300. For example, O-ring 310 may be affixed or mounted in a groove 312 disposed about interior circumferential surface 305. As should be understood, the interior circumferential surface defines fluid channel 304. O-ring 310 can be color coded according to its specific use according to any known standard such as that shown in Table 1, above, for example. By having O-ring 310 in an interior circumferential surface of fluid channel 304, O-ring 310 should not require any servicing due to the fact that female socket connector 300 is a disposable unit. Instead, a used female socket connector 300 may be replaced with another female socket connector 300.

Male connector portion 210 further includes a recessed portion 212 on male connector portion 210, adjacent to flange 208. The recessed portion 212 includes a lip 213. The male connector portion 210 mates with fluid channel 304 of female socket connector 300 by a connect/disconnect mechanism 306 engaging with recessed portion 212. For example, connect/disconnect mechanism 306 can comprise a quick release mechanism which engages with recessed portion 212 such that quick release mechanism 306 will engage and lock female socket connector 300 to male connector portion 210, with lip 213 of recessed portion 212 preventing disengagement or movement of female socket connector 300. The quick release mechanism 306 can be controlled by tab 308 extending from an exterior surface of female connector portion 304.

As shown in FIG. 4B, for example, connect/disconnect mechanism 306 comprises a spring loaded mechanism or a resilient, flexible material that will automatically engage with recessed portion 212 of male connector portion 210. In this way, connect/disconnect mechanism 306 allows for quick connect and disconnect of male connector portion 210, e.g., coupling valve, to female socket connector 300. For example, connect/disconnect mechanism 306 engages with recessed portion 212 of male connector portion 210 by sliding female socket connector 300 onto male connector portion 210. As female socket connector 300 fully slides over male connector portion 210, connect/disconnect mechanism 306 will engage with recessed portion 212 of male connector portion 210 and lock female socket connector 300 to male connector portion 210.

In the locked position, pin 302 will also depress and open valve actuator pin 214 creating a fluid passage between male connector portion 210 and fluid channel 304 of female socket connector 300. This fluid passage will extend through coupling 205 of male leakage test connector 200 and hose 400, as well as coupler 314 and hose assembly 500 connected to female socket connector 300. Coupler 314 can a ribbed coupler, hydraulic coupler, etc. As should be understood by those of skill in the art, hose 400 will couple to and provide a fluid passage from male leakage test connector 200 to a pump system of a leakage test apparatus; whereas, hose 500 will provide a fluid passage to an endoscope. In this locked position, O-ring 310 will also engage with exterior, circumferential surface of male connector portion 210, providing a fluid tight connection between male leakage test connector 200 and female socket connector 300.

To disengage female socket connector 300, a user depresses tab 308 of female socket connector 300 to disengage connect/disconnect mechanism 306 from recessed portion 212 of male connector portion 210. This is followed by sliding female socket connector 300 away from male connector portion 210.

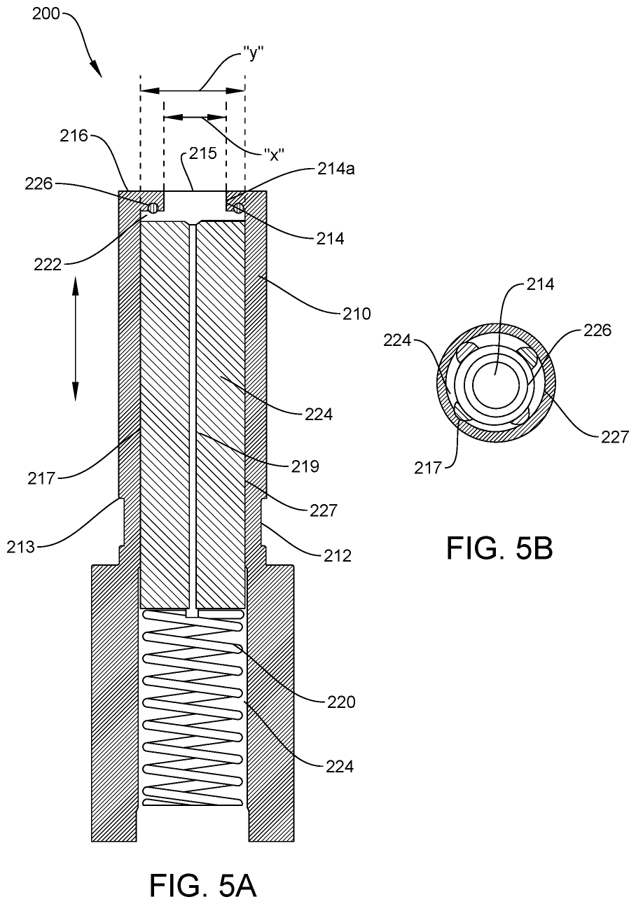

FIG. 5A depicts a partial cross-sectional view of male leakage test connector 200. FIG. 5B depicts a top down view of male leakage test connector 200 with valve actuator pin 214 comprising a tip 215 flush with top surface 216 of male leakage test connector 200. As shown, valve actuator pin 214, and more specifically, tip 215 sits flush with top surface 216 of male connector portion 210. To accomplish this feature, valve actuator pin 214 includes a stepped end portion 214a comprising a dual diameter feature of dimensions “x” and “y”. Diameter “y” is greater than diameter “x”. The fluid channel 224 also includes a stepped end portion 222 having a dual diameter of dimensions “x” and “y”, in which “y”>“x”. Valve actuator pin 214 includes a plurality of legs or ribs 217 extending from a central spine 219, below stepped end portion 214a. Two, three, four, or more legs or ribs 217 can extend along the central spine 219 at an equal spacing, e.g., 90 degrees in the case of four ribs.

A fluid channel (e.g., hollow interior portion) 224 is created between an inner, circumferential surface 227 of male connector portion 210 and the plurality of legs or ribs 217 (as shown more clearly in FIG. 5B), which provide passageways or channels that fluid may flow into and through when the valve actuator pin 214 is depressed, i.e., actuated. A spring 220 is positioned within fluid channel 224, below the plurality of legs or ribs 217. The spring 220 biases valve actuator pin 214 upwards such that wider diameter “y” of stepped end portion 214a will rest on a lower surface of walls of male leakage test connector 200 which form diameter “x”. In this way, valve actuator pin 214 will be limited in its upward movement. In this closed position, an O-ring 226 can be used to seal valve actuator pin 214 to fluid channel 224 of male connector portion 210. This will result in a fluid or air tight connection between male connector portion 210 and valve actuator pin 214.

Upon activation, e.g., by depression of the valve actuator pin 214 by pin 302 of female socket connector 300, valve actuator pin 214 will be depressed, compressing spring 220, allowing fluid passage within fluid channel 224 to exit male connector and flow into fluid channel 304 of female socket connector 300. Upon removal of female socket connector 300 from male connector portion 210, pin 302 will disengage from valve actuator pin 214 and spring 220 will force valve actuator pin 214 upwards to its closed or resting position, e.g., flush with top surface 216. The depression and upward movement of valve actuator pin 214 is depicted by double arrow in FIG. 5A.

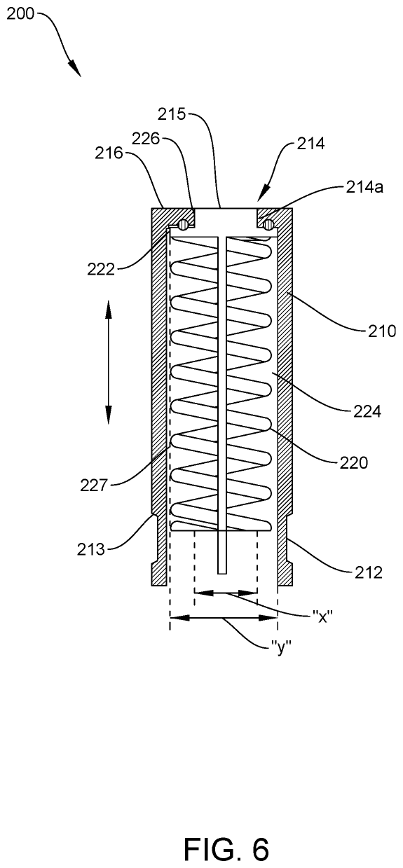

FIG. 6 depicts a partial cross-sectional view of an alternative male leakage test connector 200 with valve actuator pin 214. In this embodiment, valve actuator pin 214 also includes stepped end portion 214a comprising dual diameter feature of dimensions “x” and “y”. The fluid channel 224 also includes a stepped end portion 222 having a dual diameter of dimensions “x” and “y”, in which “y”>“x”. Valve actuator pin 214 is located and slides with a fluid channel (e.g., hollow interior portion) 224 of male connector portion 210. In this embodiment, the fluid channel 224 includes grooves 227, which provide passageways or channels for fluid flow when the valve actuator pin 214 is depressed, i.e., actuated. The spring 220 is positioned within fluid channel 224, sitting against the wider diameter “y” of stepped end portion 214. The spring 220 biases valve actuator pin 214 upwards such that wider diameter “y” of stepped end portion 214 will rest on lower surface of walls of male leakage test connector 200 which form diameter “x”. In this way, valve actuator pin 214 will be limited in its upward movement. In this closed position, O-ring 226 can be used to seal valve actuator pin 214 to fluid channel 224 of male connector portion 210. This will result in a fluid or air tight connection between male connector portion 210 and valve actuator pin 214.

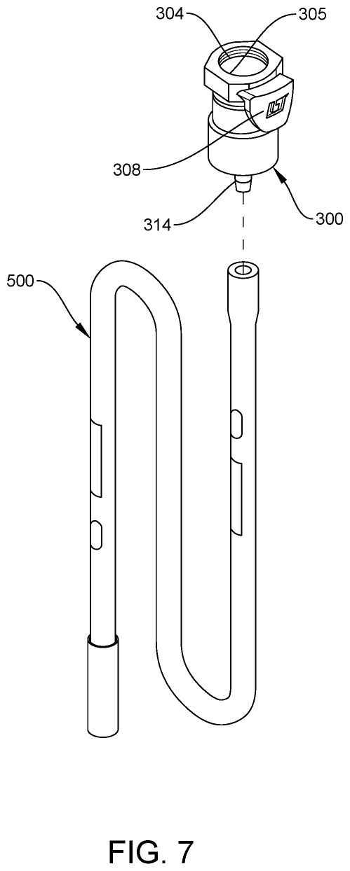

FIG. 7 depicts an exploded view of disposable female socket connector 300 and hose assembly 500. In this exploded view, it is shown that female socket connector 300 includes a coupler 314. Coupler 314 will couple to hose 500. Coupler 314 can a ribbed coupler, hydraulic coupler, etc.

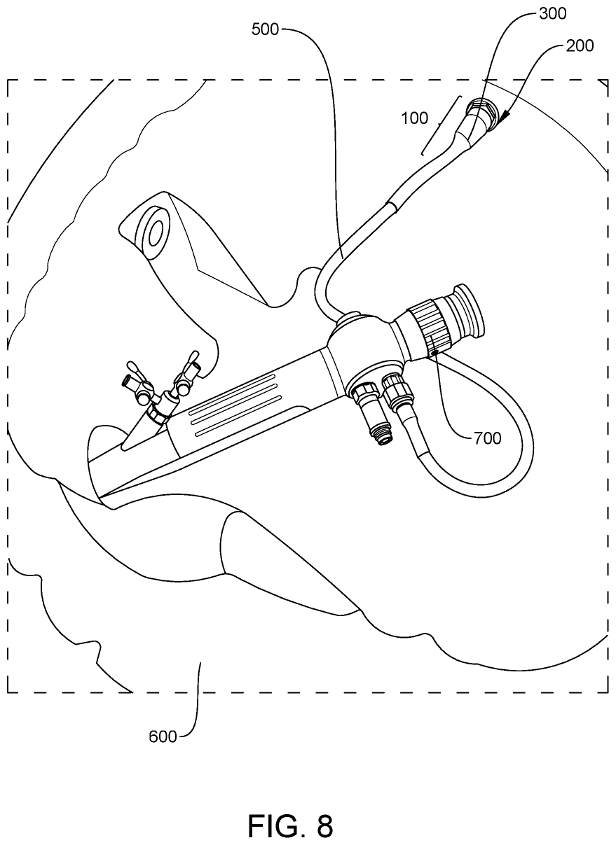

FIG. 8 depicts leakage test connector 100 connected to an endoscope 700 within a basin 600. In this representative depiction, basin 600 can be part of leakage tester apparatus, e.g., within an endoscope reprocessor, such as the EVOTECH® Cleaner and Reprocessor. The male leakage test connector 200 is fixedly attached to basin 600 by nuts 206 (not shown) and with washer 218 sealing male leakage test connector 200 to basin 600. Female socket connector 300 is mated and locked to male connector portion 210, with pin 302 depressing valve actuator pin 214 to open fluid passage within the leakage test connector 100. Hose 500 provides a fluid passage from leakage test connector 100, e.g., female socket connector 300, to endoscope 700.

By virtue of the embodiments illustrated and described herein, methods and variations thereof are implemented using leakage test connector 100. By way of example, after male leakage test connector 200 is fixedly attached to basin 600 of an endoscope reprocessor, e.g., EVOTECH® Cleaner and Reprocessor, as representatively shown in FIG. 8, a user slides female socket connector 300 over male connector portion 210 until connect/disconnect mechanism 306 engages and locks with recessed portion 212 of male connector portion 210. In the locked state, pin 302 will depress valve actuator pin 214 by compressing spring 220. This will open valve actuator pin 214 by moving open valve actuator pin 214 downward within fluid channel (hollow interior portion) 224 of male connector portion 210, opening a fluid passage from a pump leakage tester apparatus to an endoscope connected to hose 500. In the locked position, seal 310 will also engage an outer surface of male connector portion 210 to create a fluid or air tight connection. To disengage female socket connector 300, user depresses tab 308 of female socket connector 300 to disengage connect/disconnect mechanism 306 from within recessed portion 212 of male connector portion 210, followed by removal of female socket connector 300. The female socket connector 300 can be disposed of after its useful service life, e.g., after a single use.

Any of the examples or embodiments described herein may include various other features in addition to or in lieu of those described above. The teachings, expressions, embodiments, examples, etc., escribed herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

We claim:

1. A leakage test connector comprising:
a male connector having an outer circumferential surface;
a female socket connector that mates to the male connector, the female socket connector comprising a fluid passage; and
an O-ring mounted about an inner circumferential surface of the fluid passage of the female socket connector, in which the male connector lacks an O-ring on the outer circumferential surface in an unmated state with the female socket connector, such that, upon mating the male connector to the female socket connector, the O-ring mounted about the inner circumferential surface of the fluid passage of the female socket connector contacts the outer circumferential surface of the male connector.

2. The leakage test connector of claim 1, in which the male connector comprises a valve actuator pin.

3. The leakage test connector of claim 2, in which the valve actuator pin comprises a spring mechanism.

4. The leakage test connector of claim 3, in which the female socket connector comprises a pin, such that upon mating to the male connector, the pin depresses the valve actuator pin and compresses the spring.

5. The leakage test connector of claim 1, in which the female socket connector includes a groove in the interior circumferential surface and the O-ring is positioned within the groove.

6. The leakage test connector of claim 1, in which the male connector and the female socket connector both comprise hose couplings.

7. The leakage test connector of claim 6, in which the hose couplings comprise ribbed couplings or a hydraulic coupler.

8. The leakage test connector of claim 1, in which the male connector comprises a valve actuator pin having a top surface disposed flush with a tip of the male connector.

9. The leakage test connector of claim 8, in which the valve actuator pin comprises a plurality of ribs that define part of a fluid channel with an interior surface of the male connector.

10. The leakage test connector of claim 9, in which the fluid channel comprises space between each rib of the plurality of ribs and the interior surface of the male connector.

11. A leakage test connector comprising:
a male connector comprising a male connection portion comprising a fluid channel;
a valve actuator pin disposed within the fluid channel, the valve actuator pin including a top surface disposed flush with a tip of the male connector; and
a female socket connector comprising a fluid passage aligned with the fluid channel of the male connection portion.

12. The leakage test connector of claim 11, in which the male connector lacks an O-ring on its outer circumferential surface.

13. The leakage test connector of claim 12, further comprising an O-ring mounted to an inner surface of the fluid passage of the female socket connector.

14. The leakage test connector of claim 13, in which the female socket connector is disposable.

15. The leakage test connector of claim 11, in which the valve actuator pin comprises a spring mechanism.

16. The leakage test connector of claim 15, in which the female socket connector comprises a pin, such that upon mating to the male connector, the pin depresses the valve actuator pin and compresses the spring.

17. The leakage test connector of claim 16, in which the male connector and the female socket connector both comprise hose couplings.

18. The leakage test connector of claim 16, in which the valve actuator pin comprises a plurality of ribs that define the fluid channel with an interior surface of the male connector.

19. The leakage test connector of claim 18, in which the fluid channel comprises space between each rib of the plurality of ribs and the interior surface of the male connector.

20. The leakage test connector of claim 16, in which the fluid channel comprises grooves defined within an interior surface of the male connector.
